SEQUENCE LISTING (5 sequences)

SEQ ID NO: 1 - protein, 133 aa, Artificial Sequence
  The artificial sequence is synthetic.
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 2 - protein, 585 aa, Artificial Sequence [sequence omitted]

SEQ ID NO: 3 - DNA, 2187 bp, Artificial Sequence
  The artificial sequence is synthetic.
gcaccaactagcagcagcactaagaagacccagctgcaactcgaacatctgctgctcgacctgcaaatgatcctgaatgggattaacaactataagaatcccaagctcactcggatgctcaccttcaagttctacatgcccaagaaagctactgaactcaaacatctgcaatgtctcgaagaggaactgaagccactggaagaggtgctgaatctggcccaatctaagaatttccacctgaggcctagggatctgataagcaatatcaacgtgattgtgctggagctgaagggttctgaaacaacctttatgtgtgagtacgcagacgaaaccgccaccatcgtcgagttcctcaatcggtggatcaccttcgctcagagcatcatctccacactcacaggaggcggtgggagtggtggtggagggtcagacgctcacaagagcgaggtggctcatcggttcaaagacctcggcgaggagaacttcaaggccctggtcctgatcgcctttgcccagtacctgcaacagtgtccctttgaagatcatgtcaagctcgtcaacgaggtgactgagttcgccaagacatgcgtcgcagacgagagtgccgagaattgcgacaaatccctccacaccctgtttggcgataagctctgtactgtcgctactctgagggagacatacggcgagatggctgactgctgcgccaagcaggagcccgagcggaatgaatgctttctgcaacacaaagacgacaatcctaatctgccaaggctcgtcagaccagaggtggatgtgatgtgtaccgcctttcatgataatgaggaaacctttctcaagaagtatctgtatgaaattgctagaaggcacccatacttctatgctcctgagctgctgttcttcgccaaacgctacaaggctgctttcactgaatgctgccaggcagccgacaaagcagcctgcctcctgccaaagctcgacgaactccgggacgaagggaaggcctcaagtgccaagcagagactgaagtgcgcaagcctccagaaattcggagaaagggcattcaaggcttgggcagtggcaaggctgtcccagaggtttcctaaggccgaatttgccgaagtctctaaactggtcacagacctgaccaaagtgcataccgaatgttgccacggcgacctcctggagtgcgctgacgatagggcagacctggccaaatacatttgcgagaaccaggatagtataagtagcaaactcaaggagtgctgtgagaagcctctcctggagaagtcccactgcatcgccgaagtggagaacgatgagatgccagctgacctccctagcctggcagccgactttgtcgaatcaaaggacgtctgcaagaattacgccgaggcaaaggatgtgttcctgggaatgtttctgtacgagtatgccagaaggcaccctgactattcagtcgtgctgctgctgcgcctggccaagacttatgaaactaccctggagaagtgttgcgcagctgccgacccacacgagtgttatgccaaggtgttcgatgagttcaagccactcgtcgaggagcctcagaacctgatcaagcagaactgcgaactcttcgagcagctgggagagtacaaattccagaacgccctgctggtgcggtacaccaagaaggtgccacaggtgtctacacctacactggttgaagtgtccagaaacctcggcaaggtgggaagcaaatgctgcaaacatcccgaggcaaagcggatgccatgcgcagaagattacctctctgtggtgctcaatcaactgtgcgtgctgcacgagaagacccccgtcagtgacagagtgaccaaatgctgcactgagagtctggtcaatagacggccatgcttcagtgccctggaggtggacgaaacatacgttcccaaagagttcaacgccgaaactttcacctttcatgctgacatttgtaccctgtcagagaaggagcggcagattaagaaacaaactgccctggtggaactggtgaaacacaagcctaaggcaaccaaagaacaactgaaggccgtcatggacgatttcgctgccttcgtcgagaagtgttgtaaggctgacgacaaggagacatgtttcgccgaagagggcaagaaactggttgctgccagccaagctgcactgggactgtga

SEQ ID NO: 4 - protein, 728 aa, Artificial Sequence
  The artificial sequence is synthetic.
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 5 - protein, 16 aa, Artificial Sequence
  The artificial sequence is synthetic.
MPLLLLLPLLWAGALA

(19) United States
(12) Patent Application Publication
(10) Pub. No.: US 2024/0287152 A1
(43) Pub. Date: Aug. 29, 2024

(54) LONG-ACTING RECOMBINANT HUMAN INTERLEUKIN-2 FUSION PROTEIN, AND PREPARATION METHOD THEREFOR AND APPLICATION THEREOF

(71) Applicant: BEIJING VDJBIO CO., LTD., Beijing (CN)

(72) Inventors: Ziqiang LI, Beijing (CN); Xinsheng TIAN, Beijing (CN); Jianwei CHENG, Beijing (CN); Rong MA, Beijing (CN); Yanjing JIANG, Beijing (CN); Yiping SUN, Beijing (CN); Xiaorui ZHANG, Beijing (CN); Tingting ZHAO, Beijing (CN); Jun ZHANG, Beijing (CN)

(21) Appl. No.: 18/686,504

(22) PCT Filed: Sep. 29, 2021
(86) PCT No.: PCT/CN2021/121808

§ 371 Date: Feb. 26, 2024

(30) Foreign Application Priority Data
Aug. 25, 2021 (CN) 202110982310.8

(51) Int. Cl. C07K 14/55; A61K 38/00; C07K 14/765; C12N 15/85

(52) U.S. Cl. CPC C07K 14/55; C07K 14/765; C12N 15/85; A61K 38/00; C07K 2319/02; C07K 2319/31; C12N 2800/107; C12N 2800/22

(57) ABSTRACT

A fusion protein of interleukin-2 and human serum albumin. By electroporating a plasmid carrying interleukin-2 and human serum albumin fusion gene into a CHO cell, a CHO monoclonal cell line stably and efficiently expressing a human recombinant protein is obtained. The monoclonal cell line can secrete and express a fusion protein of interleukin-2 and human serum albumin. The fusion protein can prolong the plasma half-life of human interleukin-2, and can be used for preparing human interleukin-2 drugs or drugs for treating various diseases such as tumors and immunodeficiency diseases.

### TECHNICAL FIELD

The present invention relates to the field of biopharmaceuticals, specifically to a fusion protein of human interleukin-2 and human serum albumin, and coding genes and application thereof.

### BACKGROUND

Interleukin-2 (IL-2) is a glycoprotein of 15.5 kD produced by T cells and NK cells, which plays an important role in the immune response of bodies. (Human) Interleukin-2 ((h)IL-2) is composed of 133 amino acid residues (SEQ ID NO: 1), with an intra-chain disulfide bond and a cysteine located at the 125th amino acid residue of mature protein. This cysteine is prone to form mismatched disulfide bonds with the other two cysteine residues, and IL-2 with mismatched disulfide bonds is inactive. Mutating the cysteine to serine or alanine can avoid this problem without altering the activity. IL-2 mainly acts on immune cells, including T cells, large granular lymphocytes, monocytes and B cells, promoting cell proliferation and secretion of cytokines. IL-2 has anti-tumor, anti-microbial infection, and immunomodulatory effects in vivo. IL-2, either used alone or in combination with IFN-α, monoclonal antibodies, LAK cells, and the like for clinical use, has a certain therapeutic effect on renal cell carcinoma, metastatic melanoma, lymphoma, and the like. It also has therapeutic effects on colon cancer and can improve the tolerance of tumor patients to radiotherapy and chemotherapy. At present, IL-2 is widely used in clinical practice for the treatment of tumors, viral hepatitis, and the like with significant therapeutic effects. However, the above-mentioned diseases generally have a longer course of disease and therefore require long-term use of IL-2. The plasma half-life of IL-2 is only about 2 hours, and high doses and frequent injections are required to maintain its efficacy. This not only greatly increases the cost of treatment, but also increases the patient's pain and side effects.

People extend the half-life of drugs by constructing fusion proteins to increase their molecular weight. Serum albumin is an important component of plasma, and is also a carrier of many endogenous factors and exogenous drugs. Under normal circumstances, it cannot pass through the glomerulus easily. Human serum albumin (HSA) is a protein composed of 585 amino acid residues (SEQ ID NO: 2), with a molecular weight of approximately 66.5 KD and a plasma half-life of over 2 weeks.

Patent application No. CN104789594A discloses fed-batch culture of stable cell lines in 3 L of shake flask, which was carried out in 80 mL of seed solution having a density of 4.0×10⁶ cells/mL. The cell density reached to 6.4×10⁶ cells/mL on Day 3. After Day 3, the temperature was lowered and the cell density did not change significantly, and the final protein concentration was 118 mg/L. The high expression level of cell line is a basic requirement for the production of biomacromolecules such as antibodies in recent years.

Therefore, there is a need of a fusion protein of human interleukin-2 and serum albumin with higher expression levels and longer efficacy.

### SUMMARY OF THE INVENTION

The objective of the present invention is to provide a fusion protein of human interleukin-2 and serum albumin that can prolong the plasma half-life of human interleukin-2, and the coding gene thereof.

In order to achieve the purpose of the present invention, the present invention provides a long-acting recombinant fusion protein of human interleukin-2 and human serum albumin and a preparation method therefor. The fusion protein of human interleukin-2 and human serum albumin is expressed using a mammalian eukaryotic expression system, and the two are directly connected or connected via a linker peptide.

In the first aspect, the present invention provides an expression system that expresses a fusion protein of human interleukin-2 and human serum albumin, wherein the fusion protein comprises:
a human interleukin-2 or a variant thereof, wherein the human interleukin-2 or a variant thereof comprises: an amino acid sequence set forth in SEQ ID NO: 1; an amino acid sequence set forth in SEQ ID NO: 1 having one or more amino acids substitution, deletion and/or addition and meanwhile retaining equivalent functions; or an amino acid sequence having at least 90% sequence identity to the amino acid sequence set forth in SEQ ID NO: 1; and
a human serum albumin or a variant thereof, wherein the human serum albumin or a variant thereof comprises: an amino acid sequence set forth in SEQ ID NO: 2; an amino acid sequence set forth in SEQ ID NO: 2 having one or more amino acids substitution, deletion and/or addition and meanwhile retaining equivalent functions; or an amino acid sequence having at least 90% sequence identity to the amino acid sequence set forth in SEQ ID NO: 2.

The expression system according to the present invention is a CHO cell; preferably, the expression system is a CHO-K1 cell.

It is unexpectedly discovered in the present invention that when using CHO cells, especially the CHO-K1 cell line, the cell expression level is very high, reaching up to 4 g/L; moreover, the expressed fusion protein has a high activity and strongly promotes the proliferation of specific cells such as NK cells and/or T cells in vitro.

In some embodiments, the fusion protein of human interleukin-2 and human serum albumin comprises an amino acid sequence set forth in SEQ ID NO: 1 and an amino acid sequence set forth in SEQ ID NO: 2.

The amino acid sequence set forth in SEQ ID NO: 1 is human interleukin-2 composed of 133 amino acid residues, and the amino acid sequence set forth in SEQ ID NO: 2 is human serum albumin composed of 585 amino acid residues.

In some embodiments, the amino acid at position 125 of SEQ ID NO: 1 in the fusion protein is not cysteine, and preferably, the amino acid at position 125 is substituted with serine or alanine.

In some embodiments, the human interleukin-2 or a variant thereof is directly connected to the human serum albumin or a variant thereof, or the human interleukin-2 or a variant thereof is connected to the human serum albumin or a variant thereof via a linker peptide.

In the fusion protein according to the present invention, in order to create a large gap between the two moieties contained in the fusion protein and maximize the binding of the human interleukin-2 moiety to the interleukin-2 receptor, a linker peptide is provided between the human interleukin-2 or a variant thereof and the human serum albumin or a variant thereof. Preferably, the linker peptide is represented by a general formula (GₙS)ₘ, wherein n and m are an integer from 1 to 10, respectively; more preferably, n is an integer from 1 to 4, and m is an integer from 0 to 3.

In some embodiments, the N-terminus of the fusion protein carries a signal peptide; preferably, the signal peptide is a secretary signal peptide CD33; more preferably, the signal peptide sequence is MPLLLLLPLLWAGALA, as set forth in SEQ ID NO: 5.

In some embodiments, the fusion protein is encoded by the nucleotide sequence set forth in SEQ ID NO: 3.

In the present invention, based on the gene sequences of human interleukin-2 and human serum albumin published by NCBI, gene sequence optimization is carried out according to mammalian codon preference. The optimized gene sequence of a fusion protein is constructed onto an expression vector, transfected into a host cell, expressed in the host cell, isolated and purified to obtain a target protein.

The optimized gene sequence of the fusion protein according to the present invention is added with a signal peptide at its N-terminus, preferably added with a secretary signal peptide CD33 sequence, constructed onto an expression vector, and the obtained vector is transfected into a host cell, expressed in the host cell, isolated and purified to provide a target protein.

In the present invention, the optimized gene sequence carries a signal peptide at its N-terminus, and the gene sequence that is constructed onto the expression vector is set forth in SEQ ID NO: 3.

In some embodiments, the fusion protein of human interleukin-2 and human serum albumin has an amino acid sequence set forth in SEQ ID NO: 4.

Wherein, SEQ ID NO: 4 is a protein composed of 728 amino acid residues. Position 1 from the N-terminus to position 133 correspond to a coding sequence for human interleukin-2, position 134 from the N-terminus to position 143 correspond to a coding sequence for a linker peptide Gly Gly Gly Gly Ser Gly Gly Gly Gly Ser, and position 144 from the N-terminus to position 728 correspond to a coding sequence for human serum albumin.

In the second aspect, the present invention provides the use of the expression system in the preparation of a medicament for the treatment of tumors, hepatitis, pneumonia, or immunodeficiency diseases.

### Beneficial Effects of the Present Invention

The present invention discloses an expression system expressing a fusion protein of interleukin-2 and human serum albumin. By electroporation of the plasmid with the fusion gene of interleukin 2 and human serum albumin into CHO cells, a CHO monoclonal cell line with stable and efficient expression of human recombinant protein was obtained. The monoclonal cell line according to the present invention can secrete a fusion protein expressing interleukin-2 and human serum albumin. This fusion protein can prolong the plasma half-life of human interleukin-2 and can be used to prepare human interleukin-2 drugs or drugs for treating various diseases such as tumors and immunodeficiency diseases.

The present invention achieves high expression levels not only through clone screening, but also by adding a signal peptide to N-terminus of the optimized fusion protein gene sequence during cell construction, which is also one of the reasons for high expression levels.

The cell line of the present invention not only has a high expression level of up to 4 g/L, but also expresses fusion proteins with high activity and having a strong promoting effect on proliferation of NK cells and/or T cells.

### DESCRIPTION OF DRAWINGS

FIG. 1 shows reductive electrophoresis analysis results of a well plate culture supernatant of IL-2-HSA/CHOK1 clone screening, with 20 μL of supernatant sample added.

FIG. 2 shows non-reductive electrophoresis analysis results of the fed-batch culture D13 supernatant of IL-2-HSA/CHOK1 clone screening, with 5 μL of supernatant sample added.

FIG. 3 shows non-reductive electrophoresis analysis results of the fed-batch culture D13 supernatant of IL-2-HSA/CHOK1 clone #9 subjected to monoclonal screening, with 3 μL of supernatant sample added.

FIG. 4 shows non-reductive electrophoresis analysis results of the fed-batch culture D13 supernatant of IL-2-HSA/CHOK1 clone #9-6 subjected to monoclonal screening, with 2 μL of supernatant sample added.

FIG. 5 shows a kinetics curve of IL-2-HSA/CHOK1 cell culture. Fed-batch culturing was carried out in a 125 mL shake flask with an initial cell density of 0.3×10⁶ cells/mL, an initial culture volume of 25 mL, and a maximum live cell density of 19.3×10⁶ cells/mL.

FIG. 6 shows a protein expression kinetics curve of the supernatant D7-D13 obtained from IL-2-HSA/CHOK1 cells through low-density inoculation culture process.

FIG. 7 shows non-reductive electrophoresis analysis results of IL-2-HSA/CHOK1 cell expression supernatant, with 1 μL of supernatant sample added.

FIG. 8 shows a kinetics curve of IL-2-HSA/CHOK1 cell culture. Fed-batch culture was carried out in a 1 L shake flask with an initial cell density of 2×10⁶ cells/mL, an initial culture volume of 300 mL and a maximum live cell density of 16×10⁶ cells/mL.

FIG. 9 shows a protein expression kinetics curve of the supernatant DO-D10 obtained from IL-2-HSA/CHOK1 cells through a high-density inoculation culture process.

FIG. 10 shows non-reductive electrophoresis analysis results of IL-2-HSA/CHOK1 cell expression supernatant, with 5 μL of supernatant sample added.

FIG. 11 shows a curve of NK-92 proliferation stimulated by IL-2-HSA.

FIG. 12 shows a curve of CTLL-2 proliferation stimulated by IL-2-HSA.

### BEST MODE OF THE INVENTION

The present invention is further illustrated through the examples below. It should be understood that the examples of the present invention are only intended to illustrate the present invention but not to limit it. Any simple improvement made to the present invention under the concept of the present invention is within the scope of protection claimed by the present invention.

### Example 1: Construction of Stable Transfected Cell Lines

On the day before transfection, the density of CHO-K1 cells was adjusted to 0.5×10⁶ cells/mL. On the day of transfection, high concentration endotoxin-free plasmids that had undergone linear treatment were prepared, the cell density and viability of CHO-K1 cells were measured, ensuring that the cell viability was greater than 97%. After the CHO-K1 cells were washed twice with CD CHO medium, an electroporation transfection reaction system was prepared: 700 μL of cell suspension and 40 μg of plasmid were mixed well and transferred into a 4 mm of electrode cup. The electrode cup was placed into an electroporator and the shock parameters were set to 300 V, 1000 μF. After one electric shock, the shocked cell suspension was transfer into preheated fresh CD CHO medium, and incubated at 37° C. for 20 minutes. The incubated cell suspension was inoculated evenly into a 96-well plate, transfected for 24 hours, pressurized, and added with CD CHO medium containing methionine sulfoximine (MSX), under the final screened pressure of 25-50 μM MSX, with 5% CO₂, at 37° C. for static culture.

### Example 2: Screening of High Expression Monoclonal Cell Lines

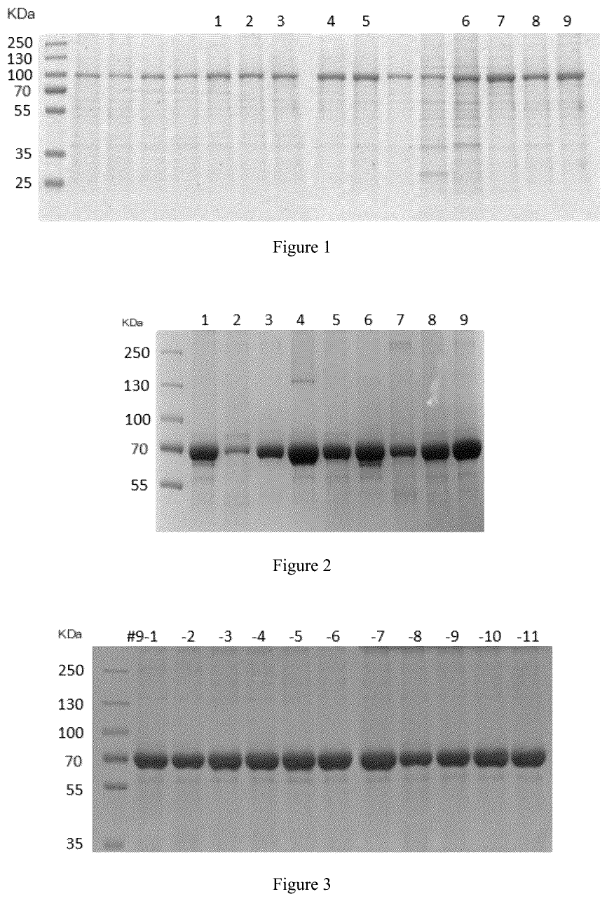

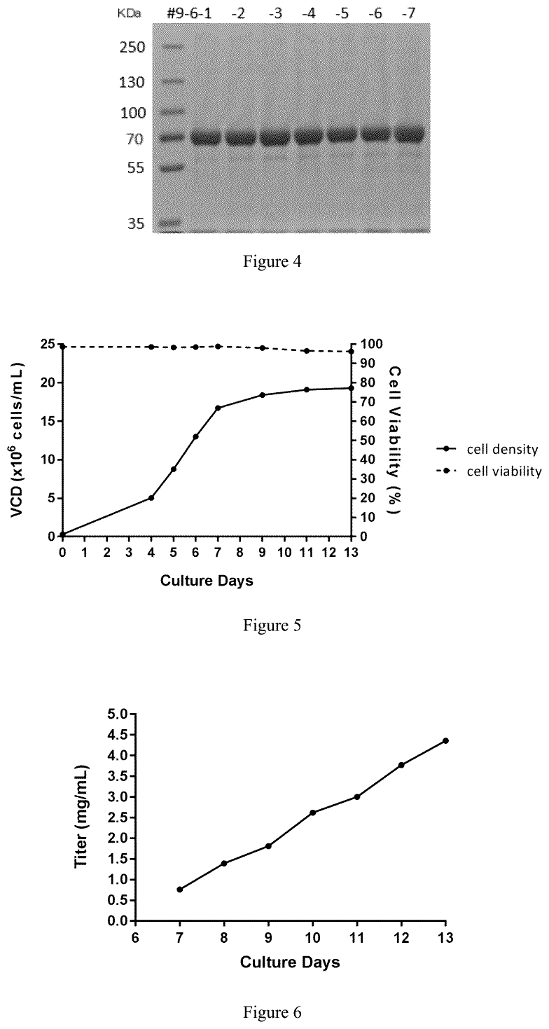

After growing to an appropriate size in the 96-well plate, the monoclonal cell lines were selected and all clones were transferred to a new 96-well plate, incubated in 5% CO₂ at 37° C. for static culture. After the cells in the well were fully grown, the supernatant was taken from the well plate for reductive electrophoresis to detect the expression of fusion proteins. Nine clones with the highest expression levels were selected (see FIG. 1) and gradually expanded to shake flask culture. Nine clones were subjected to fed-batch culture in 25 mL shake flasks, and the culture supernatant was harvested and identified by non-reductive electrophoresis (see FIG. 2). The cell line #9 with the best expression level was selected. Monoclonal cell lines were screened using limited dilution method for cell line #9. A 96-well plate was inoculated with 0.3 cells/well to obtain 11 highly expressed cell lines, which were subjected to fed-batch culture in 25 mL shake flasks, and the supernatant was identified by non-reductive electrophoresis (see FIG. 3). The cell line #9-6 with the best expression was selected. Monoclonal cell lines were screened again using limited dilution method for cell line #9-6, resulting in 7 high expressed cell lines, which were subject to fed-batch culture in 25 mL shake flasks and the supernatant was identified by non-reductive electrophoresis (see FIG. 4). Cell line #9-6-7 with good stability and high expression levels was selected as stable and high expression cell line IL-2-HSA/CHOK1.

### Example 3: Fed-Batch Culture of Stable Cell Line in 125 mL Shake Flask

On the day (recorded as DO) when IL-2-HSA/CHOK1 cells were started to be cultured for expression, 25 mL of minimum medium containing 25˜50 μM MSX were inoculated into a 125 mL of shake flask at a density of 0.3×10⁶ cells/mL, and cultured with 5% CO₂ at 37° C. on a shaker at 135 rpm. On D4 of inoculation, the samples were taken and counted, and the culture temperature was decreased to 33° C. when the cell density reached to 10×10⁶ cells/mL. On D5, fed-batch culture was carried out and the glucose concentration was controlled to 3˜4 g/L. On D13 of culture, the culture was terminated, the supernatant of the cell culture solution was collected, and the expression level of the fusion protein was measured to be 4.36 mg/mL.

A kinetics curve of IL-2-HSA/CHOK1 cell culture is shown in FIG. 5. It can be seen from FIG. 5 that in the early stage of culture, the cells were in a logarithmic growth stage, with a rapid increase in density; in the later stage of culture, the cells entered a protein production stage, and the cell density tended to stabilize. The maximum live cell density reached to 19.3×10⁶ cells/mL.

A kinetics curve of IL-2-HSA/CHOK1 cell supernatant expression level is shown in FIG. 6. It can be seen from FIG. 6 that from D7 to D13, with the increase of culture days, the protein expression level of the cell showed a gradually increasing trend. The expression level on D13 was 4.36 mg/mL.

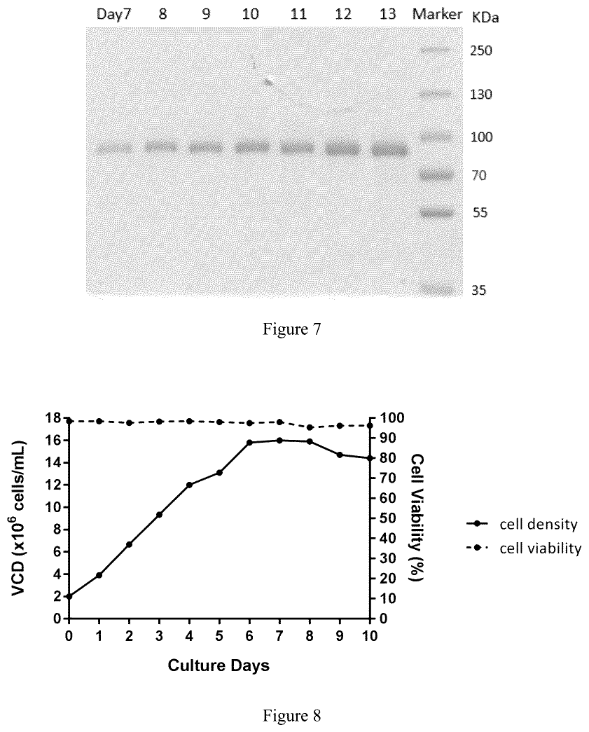

A non-reductive electrophoresis analysis of IL-2-HSA/CHOK1 cell expression supernatant is shown in FIG. 7. It can be seen from FIG. 7 that from D7 to D13, with the increase of culture days, the protein expression level of the cell showed a gradually increasing trend. The target protein had a single band without impurities.

### Example 4: Fed-Batch Culture of Stable Cell Line in 1 L of Shake Flask

IL-2-HSA/CHOK1 cells were inoculated into a 50 mL of shaking flask at a density of 2×10⁶ cells/mL, and cultured with 5% CO₂ at 37° C. on a shaker at 135 rpm. The samples were taken and counted daily to observe cell status, and the minimum medium containing 25˜50 μM MSX was supplemented, with daily adjustment of cell density to 2×10⁶ cells/mL until the volume of the cell culture solution reached to 300 mL. The minimum medium was stopped supplementing, and the culture was continued, at which point it was recorded as DO. The samples were taken and counted daily and 1 mL of culture supernatant was retained. The culture temperature was decreased to 33° C. when the cell density reached to 6×10⁶˜7×10⁶ cells/mL. On D2, the fed-batch culture was started and the glucose concentration was controlled to 3 g/L. On D10 of culture, the culture was terminated, the supernatant of the cell culture solution was collected, and the protein expression level of the supernatant was measured from DO to D10. The expression level of fusion protein in the supernatant harvested on D10 was measured to be 3.12 mg/mL.

A kinetics curve of IL-2-HSA/CHOK1 cell culture is shown in FIG. 8. It can be seen from FIG. 8 that in the early stage of culture, the cells were in a logarithmic growth stage, with a rapid increase in density; in the later stage of culture, the cells entered a protein production stage, and the cell density tended to stabilize. The maximum live cell density reached to 16×10⁶ cells/mL.

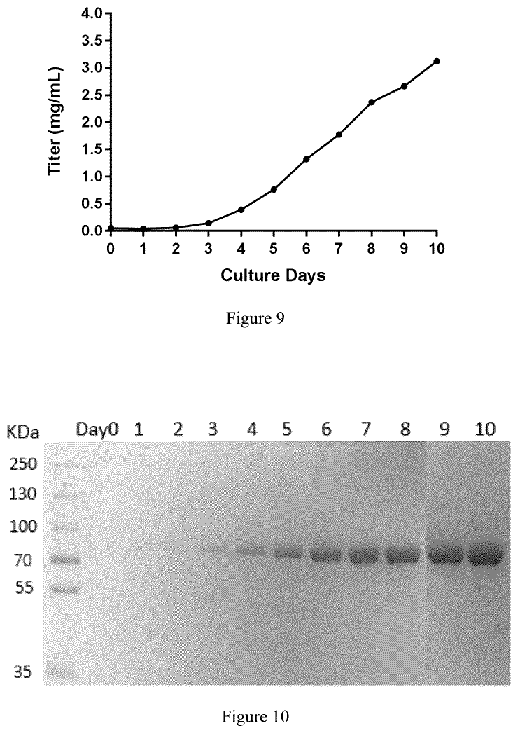

A kinetics curve of IL-2-HSA/CHOK1 cell supernatant expression level is shown in FIG. 9. It can be seen from FIG. 9 that with the increase of culture days, the protein expression level of the cell showed a gradually increasing trend.

A non-reductive electrophoresis analysis of IL-2-HSA/CHOK1 cell expression supernatant is shown in FIG. 10. It can be seen from FIG. 10 that with the increase of culture days, the protein expression level of the cell showed a gradually increasing trend. The target protein had a single band and no impurities.

Compared with the fed-batch culture in shake flask in the prior art, the cell density and cell survival rate of the present invention are very high, as shown in FIG. 8. The cell density reaches to 14×10⁶˜16×10⁶ cells/mL on D6 to D10, and the cell protein expression level of the present invention is also very high, as shown in FIG. 9. The expression level on D10 is 3.12 mg/mL. In the present application, when the cell density is 14×10⁶˜16×10⁶ cells/mL, the expression level is also high, indicating that the cell activity is still good at such a cell density.

### Example 5: Activity Experiment 1 of Fusion Protein: Detection of Effect of IL-2-HAS on NK-92 Cell Proliferation

In this example, NK-92 cells (ATCC® CRL-2407™, IL-2 dependent NK cell line derived from peripheral blood monocytes of a 50 year-old male patient with malignant non-Hodgkin's lymphoma) were utilized to evaluate the biological activity of IL-2-HSA.

(1) Cryopreserved NK-92 cells were taken out from liquid nitrogen tank and resuscitated for culture until the logarithmic growth phase.

(2) Sufficient cells were collected by centrifugation, resuspended in complete culture medium without IL-2, and cultured under starvation for 24 hours.

(3) The starved NK-92 cells were centrifuged, resuspend in complete culture medium without IL-2, and counted; the cell density was adjusted to 5×10⁵ cells/mL, and the cells were added into the 96-well plate at a volume of 90 μL per well.

(4) Preparation of sample solution: IL-2-HSA sample and rhIL-2 (R&D, Cat. No. 202-IL) were pre-diluted to 50.67 nM with the medium, and diluted to 9 concentrations at a 4-fold gradient, added to corresponding wells of the 96-well plate at 10 μL/well, with three wells for each concentration. For the negative control group, the medium was added at 10 μL/well correspondingly and mixed well.

(5) After culturing in 5% CO₂ at 37° C. for 72 hours, the melted and mixed MTS detection reagent was added to the above 96-well plate at 20 μL/well, shook well in an oscillator, and then incubated in a 37° C., 5% CO₂ cell culture incubator for 1 to 4 hours.

(6) After incubation, shook and mixed well; the absorbance was measured at a wavelength of 490 nm using a microplate reader.

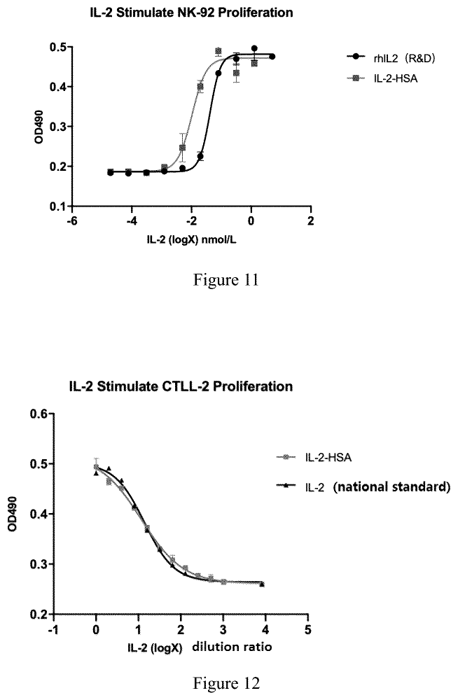

(7) The data was analyzed using GraphPad Prism 8 software, with the logarithm of drug concentration X as the horizontal coordinate and OD₄₉₀ as the vertical coordinate. The drug action curve was fitted with four parameters. The EC₅₀ values obtained are shown in Table 1, and the proliferation curve is shown in FIG. 11.

TABLE 1

| | IL-2 (R&D) | IL-2-HSA |
|---|---|---|
| EC₅₀ (nM) | 0.0420 | 0.01047 |

It can be seen from FIG. 11 that IL-2-HSA induced NK-92 cell growth in a dose-dependent manner. Under the experimental condition, the activity of IL-2-HSA in stimulating NK-92 proliferation was better than that of equimolar rhIL-2 (about 4 times).

### Example 6: Activity Experiment 2 of Fusion Protein: Detection of Effect of IL-2-HAS on CTLL-2 Cell Proliferation

In this example, CTLL-2 cells (ATCC® TIB™, mouse cytotoxic T lymphocyte cell line, IL-2 dependent) were utilized to evaluate the biological activity of IL-2-HSA.

Referring to the human interleukin-2 biological activity assay method (CTLL-2 cell/MTT colorimetric method) in the Chinese Pharmacopoeia, CTLL-2 cells were inoculated into 96-well plate at a density of 30000 cells/well. A series of gradient diluted national standards and IL-2-HSA were added, and cultured in 5% CO₂ at 37° C. for 18 to 24 hours. Then, MTS detection reagent was added and incubated for 1 to 4 hours. After shaking and mixing, the absorbance was measured at a wavelength of 490 nm using a microplate reader. The data was analyzed using GraphPad Prism 8 software, with the logarithm of dilution X as the horizontal coordinate and OD₄₉₀ as the vertical coordinate. The drug action curve was fitted with four parameters. The EC₅₀ values obtained are shown in Table 2, and the proliferation curve is shown in FIG. 12.

The biological activity of IL-2-HSA is calculated using the following formula:

$$biological activity of test sample \frac{IU}{mL} = biological activity of standard IU / mL \times \frac{pre - dilution ratio of test sample}{pre - dilution ratio of standard} \times \frac{\begin{matrix}dilution ratio of test sample equivalent \\ to the half effective amount of standard\end{matrix}}{semi - effective dilution ratio of standard}$$

After calculation, the specific activity of IL-2-HSA was 8.38×10⁶ IU/mg, comparable to the specific activity (not less than 1×10⁷ IU/mg) of IL-2 as specified in the pharmacopoeia, but the molecular weight difference between the two was about 5 times, thus the specific activity of IL-2-HSA was higher.

TABLE 2

| | IL-2 (National Standard) | IL-2-HSA |
|---|---|---|
| EC₅₀ (dilution ratio) | 13.9 | 11.68 |

The examples listed in the present invention are only the preferred technical solutions of the present invention. The scope of protection of the present invention is not limited to the above-mentioned examples. If any modification or deformation is carried out under the principal conditions of the present invention, it should belong to the scope of protection of the present invention.

1-10. (canceled)

11. An expression system characterized in that the expression system expresses a fusion protein of human interleukin-2 and human serum albumin, wherein the fusion protein comprises:
a human interleukin-2 or a variant thereof, wherein the human interleukin-2 or a variant thereof comprises: an amino acid sequence set forth in SEQ ID NO: 1; an amino acid sequence set forth in SEQ ID NO: 1 having one or more amino acids substitution, deletion and/or addition and meanwhile retaining equivalent functions; or an amino acid sequence having at least 90% sequence identity to the amino acid sequence set forth in SEQ ID NO: 1; and
a human serum albumin or a variant thereof, wherein the human serum albumin or a variant thereof comprises: an amino acid sequence set forth in SEQ ID NO: 2; an amino acid sequence set forth in SEQ ID NO: 2 having one or more amino acids substitution, deletion and/or addition and meanwhile retaining equivalent functions; or an amino acid sequence having at least 90% sequence identity to the amino acid sequence set forth in SEQ ID NO: 2.

12. The expression system according to claim 11, characterized in that the expression system is a CHO cell; preferably, the expression system is a CHO-K1 cell.

13. The expression system according to claim 11, characterized in that the fusion protein of human interleukin-2 and human serum albumin comprises an amino acid sequence set forth in SEQ ID NO: 1 and an amino acid sequence set forth in SEQ ID NO: 2.

14. The expression system according to claim 13, characterized in that the amino acid at position 125 of SEQ ID NO: 1 in the fusion protein is not cysteine; preferably, the amino acid at position 125 is substituted with serine or alanine.

15. The expression system according to claim 11, characterized in that the human interleukin-2 or a variant thereof is directly connected to the human serum albumin or a variant thereof, or the human interleukin-2 or a variant thereof is connected to the human serum albumin or a variant thereof via a linker peptide,
preferably, the linker peptide is represented by a general formula (GₙS)ₘ, wherein n and m are an integer from 1 to 10, respectively; more preferably, n is an integer from 1 to 4, and m is an integer from 0 to 3.

16. An expression system according to claim 11, characterized in that the N-terminus of the fusion protein carries a signal peptide;
preferably, the signal peptide is secretory signal peptide CD33.

17. The expression system according to claim 16, characterized in that the signal peptide has a sequence set forth in SEQ ID NO: 5.

18. The expression system according to claim 11, characterized in that the fusion protein is encoded by a nucleotide sequence set forth in SEQ ID NO: 3.

19. The expression system according to claim 11, characterized in that the fusion protein of human interleukin-2 and human serum albumin has an amino acid sequence set forth in SEQ ID NO: 4.

20. Use of the expression system according to claim 11 in the preparation of a medicament for the treatment of tumors, hepatitis, pneumonia, or immunodeficiency diseases.

21. A method for the treatment of tumors, hepatitis, pneumonia, or immunodeficiency diseases, comprising administering to a subject in need thereof the expression system according to claim 11.

22. A fusion protein of human interleukin-2 and human serum albumin produced by the expression system according to claim 11.

23. A method for the treatment of tumors, hepatitis, pneumonia, or immunodeficiency diseases, comprising administering to a subject in need thereof the fusion protein according to claim 22.
